(19) United States
(12) Patent Application Publication
(10) Pub. No.: US 2005/0032888 A1
(43) Pub. Date: Feb. 10, 2005

(54) Use of ascorbic acid for producing topical preparations with angiogenetic effects

(76) Inventors: Kirsten Sauermann, Letschien (DE); Ralph Schimpf, Bonnigstedt (DE); Alexander Filbry, Hamburg (DE); Roger Wepf, Hamburg (DE); Volker Schreiner, Hamburg (DE); Soren Jaspers, Hamburg (DE); Uwe Schonrock, Nahe (DE); Joachim Ennen, Hamburg (DE); Gerhard Sauermann, Gross Neuendorf (DE)

Correspondence Address: ALSTON & BIRD LLP;BANK OF AMERICA PLAZA, 101 SOUTH TRYON STREET, SUITE 4000, CHARLOTTE, NC, 28280-4000, US

(21) Appl. No.: 10/481,067

(22) PCT Filed: Jun. 14, 2002
(86) PCT No.: PCT/EP02/06598

(30) Foreign Application Priority Data
Jun. 15, 2001 (DE) 101 28 911.1

(51) Int. Cl. A61K 31/375

(52) U.S. Cl. 514/474

(57) ABSTRACT

Use of ascorbic acid for producing topical preparations with angiogenic effects.

The present invention relates to the use of ascorbic acid for producing topical preparations with angiogenic effects, in particular cosmetic and dermatological preparations.

The blood vessel system serves to provide tissues with nutrients, vitamins, and oxygen, to transport hormones from secretion organs to target organs, and to remove metabolic end products such as CO₂ to its separation organ the lung, or urea for excretion via the kidneys.

One of few tissues, which are physiologically not supplied with blood, is the epidermis. It is supplied via the microcapillaries of the dermis, which extend in young people in a regular pattern of papillae (rete pegs) into the epidermis. This architecture was determined and found by means of classical histology—excisional biopsy, fixation, coloring, and numerous sections. The quantitative histometric description is complicated or distorted by process-caused shrinkage of the tissue specimens. Nonetheless, the classic technique permitted recognizing age-caused degeneration of the microcapillary systems and flattening of the rete pegs, i.e. the boundary zone epidermis/dermis. This results in a reduced interchange of substances with the epidermis (decrease of the transportation surface). Likewise, the extent of the interdigitated surface epidermis/dermis becomes smaller, which leads to a reduced mechanical strength and elasticity of the skin.

Status and changes of the microcapillary system and the rete peg pattern can be shown, followed, and quantified on probands quickly (in minutes) by means of modern techniques, such as confocal microscopy in vivo—i.e., free of injury, repeatedly as often as desired, and with dynamic responses to actual influences—both with respect to the density of the rete pegs or microcapillaries (number per surface unit) and with respect to their height, which makes it possible to estimate the size of the exchange surface.

Antioxidants are chemical substances of a great variety of different structures, which inhibit or prevent in the substances being protected undesired changes that are caused by the action of oxygen and other oxidative processes. Antioxidants are used in particular for protecting not only organic products, namely fats and oils, but also active agents and additives, such as, for example, aromatic substances and the like. Active as antioxidants are, for example, phenols, hydroquinones, catechins, and aromatic amines, as well as their metal complexes, that are substituted with sterically impeding groups. Suitable for fats, foods, and in particular also for cosmetic and dermatological preparations are tocopherol, butylated hydroxyanisoles (BHA), butylated hydroxytoluene (BHT), octyl gallate and dodecyl gallate, as well as ascorbic acid, lactic acid, citric acid, and tartaric acid, and their salts.

Per se excellent antioxidants are selected from the group of the ascorbic acid and ascorbyl compounds.

L-ascorbic acid {(R)-5-1,2-dihydroxyethyl]-3,4-hydroxy-5-H-furan-2-on, vitamin C} is characterized by the structural formula 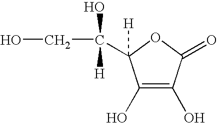
It is easily soluble in water, excellently soluble in alcohol, insoluble in ether, petroleum ether, chloroform, benzene, as well as fats and fatty oils. Ascorbic acid is an enediol and has as a reductone a strongly reducing effect. Ascorbic acid is sensitive to heat, and decomposes in particular in the presence of traces of heavy metal as well as in an alkaline environment by air and atmospheric oxygen. In a pure, dry state, however, it is relatively resistant to light, air, and heat.

It has therefore been surprising and unforeseeable for the skilled person that the use of ascorbic acid for producing topical preparations with angiogenic effects, in particular cosmetic and dermatological preparations remedies the disadvantages of the art.

Regular topical application of ascorbic acid to the skin of older probands leads to a density of rete pegs and microcapillaries that is reversibly increased to the extent of youthful skin. These structural changes manifest themselves in an improvement of both elasticity parameters of the skin (slowed-down formation of suction blisters) and moisture content and roughness of the horny layer. This juvenile anatomy expresses itself in an especially defined way in the accompanying optimized function, when responding to stress (physical stress, such as cold or dryness, aggressive agents, such as surfactants, promoters of stingings or other irritants, immunologically in a faster healing of dermatoses, wounds, or in the case of microbial infestation).

The cosmetic or dermatological preparations of the invention can have the customary composition, and be used for the treatment, care, and cleansing of the skin and/or hair, and as makeup product in decorative cosmetics. They contain ascorbic acid, preferably from 0.001 wt. % to 10 wt. %, preferably 0.05 wt. % to 5 wt. %, in particular 0.1 wt. % to 2.0 wt. % based on the total weight of the preparations.

In accordance with the invention, it is preferred to add to the used combinations of active substances of the invention, or to the cosmetic or dermatological preparations, complexing agents that contain such combinations of active substances.

Complexing agents are per se known auxiliaries of cosmetology or medical galenics. As a result of complexing disturbing metals, such as Mn, Fe, Cu, and others, it is possible to prevent, for example, undesired chemical reactions in cosmetic or dermatological preparations.

Complexing agents, in particular chelating agents form with metal atoms complexes, which represent metallacycles in the presence of one or more polybasic complexing, i.e., chelating agents. Chelates are compounds, in which a single ligand occupies more than one coordination point on a central atom. In this case, normally extended compounds are closed to rings by complexing via a metallic atom or metallic ion. The number of bound ligands depends on the coordination number of the central metal. Precondition for the chelate formation is that the compound reacting with the metal contains two or more atom groupings, which act as electron donators.

The complexing agent or agents may advantageously be selected from the group of normal compounds. Preferably, at least one substance is from of the group consisting of tartaric acid and its anions, citric acid and its anions, amino polycarboxylic acids and their anions (such as, for example, ethylenediamine tetraacetic acid (EDTA) and its anions, nitrilo-triacetic acid (NTA) and its anions, hydroxyl ethylenediamine triacetic acid (HOEDTA) and its anions, diethyleneaminepentaacetic acid (DTPA) and its anions, trans-1,2-diaminocyclo-hexane-tetraacetic acid (CDTA) and its anions).

According to the invention, cosmetic or dermatological preparations advantageously contain a complexing agent or complexing agents in an amount from 0.01 wt. % to 10 wt., preferably 0.05 wt. % to 5 wt. %, very preferably 0.1-2.0 wt. % based on the total weight of the preparations.

For their use in accordance with the invention, the cosmetic and dermatological preparations are applied, as is usual for cosmetics, in an adequate quantity to the skin and/or hair.

Cosmetic and dermatological preparations according to the invention may be present in various forms. For example, they may be a solution, an anhydrous preparation, an emulsion or microemulsion of the water-in-oil (W/O) type or oil-in-water (O/W) type, a multiphase emulsion, for example, of the water-in-oil-in-water (W/O/W) type, a gel, a solid stick, an ointment, or also an aerosol. It is also advantageous to provide ascorbic acid in an encapsulated form, for example, in collagen matrixes, and other common encapsulation materials, for example, cellulose encapsulations, in gelatin, wax matrixes, or liposomal capsules. Wax matrixes, as are disclosed in DE-OS 43 08 282, have been found especially advantageous.

For the purposes of the present invention, it is also possible and advantageous to include combinations of active constituents as used in accordance with the invention in aqueous systems or surfactant preparations for cleansing the skin and hair.

The cosmetic and dermatological preparations of the invention may contain cosmetic auxiliaries, as are commonly used in such preparations, for example, preservatives, bactericides, perfumes, antifoaming agents, dyes, pigments, which have a coloring effect, thickeners, surface-active substances, emulsifiers, softening, moisturizing and/or humectant substances, fats, oils, waxes, or other customary constituents of a cosmetic or dermatological formulation, such as alcohols, polyols, polymers, foam stabilizers, electrolytes, organic solvents, or silicone derivatives.

In particular, it is also possible to combine combinations of active constituents as are used in accordance with the invention, with other antioxidants and/or radical traps.

Advantageously, such antioxidants are selected from the group comprising amino acids (for example, glycine, histidine, tyrosine, tryptophane) and their derivatives, imidazoles (for example, urocanic acid) and their derivatives, peptides, such as D,L-carnosine, D-carnosine, L-carnosine, and their derivatives (for example, anserine), carotenoids, (for example, α-carotene, β-carotene, lycopene) and their derivatives, chlorogenic acid and its derivatives, lipoic acid and its derivatives (for example, dihydrolipoic acid), aurothioglucose, propylthiouracil, and other thiols (for example, thioredoxin, glutathione, cysteine, cystine, cystamine, and their glycosyl-, N-acetyl-, methyl-, ethyl-, propyl-, amyl-, butyl- and lauryl-, palmitoyl-, oleyl-, -linoleyl-, cholesteryl-, and glyceryl esters), as well as their salts, dilauryl thiodipropionate, distearyl thiodipropionate, thiodipropionic acid and its derivatives (esters, ethers, peptides, lipids, nucleotides, nucleosides, and salts), as well as sulfoximine compounds (for example, buthionine sulfoximine, homocysteine sulfoximine, buthionine sulfones, penta-, hexa-, heptathionine sulfoximine), in very low, tolerable dosages (for example, pmol to μmol/kg), furthermore (metallic) chelating agents (for example, a-hydroxy fatty acids, palmitic acid, phytic acid, lactoferrin), a-hydroxy acids (for example, citric acid, lactic acid, malic acid), humic acid, bile acid, bile extracts, bilirubin, biliverdin, EDTA, EGTA, and their derivatives, unsaturated fatty acids and their derivatives (for example, γ-linolenic acid, linoleic acid, oleic acid), folic acid, and their derivatives, ubiquinone and ubiquinol and their derivatives, tocopherols and derivatives (for example, vitamin E acetate), vitamin A and derivatives (vitamin A palmitate) as well as coniferyl benzoate of the benzoin resin, rutic acid and its derivatives, butyl hydroxytoluene, butyl hydroxyanisole, nordihydroguaiac resin acid, nordihydroguaiaretic acid, trihydroxy butyrophenone, uric acid and its derivatives, mannose and its derivatives, sesamol, sesamolin, zinc and its derivatives (for example, ZnO, ZnSO₄), selenium and its derivatives (for example, selenium methionine), stilbene and its derivatives (for example, stilbene oxide, trans-stilbene oxide), and the derivatives (salts, esters, ethers, sugars, nucleotides, nucleosides, peptides, and lipids) of these aforesaid active constituents, which are suitable in accordance with the invention.

The quantity of the foregoing antioxidants (one or more compounds) in the preparations is preferably 0.001-30 wt. %, very preferably 0.05-20 wt. %, in particular 1-10 wt. % based on the total weight of the preparation.

Provided vitamin E and/or its derivatives represent the additional antioxidant or antioxidants, it will be advantageous to select their respective concentrations from the range of 0.001-10 wt. % based on the total weight of the formulation.

Provided vitamin A, or vitamin A derivatives, or carotenes, or their derivatives represent the additional antioxidant or antioxidants, it will be advantageous to select their respective concentrations from the range of 0.001-10 wt. % based on the total weight of the formulation.

Emulsions according to the invention are advantageous and contain, for example, the following fats, oils, waxes, and other lipids, as well as water, and an emulsifier, as is commonly used for such a type of the formulation.

The lipid phase may advantageously be selected from the following group of substances:
mineral oils, mineral waxes;
oils, such as triglycerides of the capric or caprylic acid, furthermore, natural oils, such as, for example, castor oil;
fats, waxes, and other natural and synthetic lipids, preferably esters of fatty acids with alcohols having a low number of C atoms, for example, isopropanol, propylene glycol or glycerin, or esters of fatty alcohols with alkanoic acids having a low number of C atorms, or with fatty acids;
alkyl benzoates;
silicone oils, such as dimethyl polysiloxanes, diethyl polysiloxanes, diphenyl polysiloxanes, as well as mixed forms thereof.

The oil phase of the emulsions, oleogels, or hydrodispersions or lipodispersions for the purposes of the present invention is advantageously selected from the group of the esters of saturated and/or unsaturated, branched and/or unbranched alkanecarboxylic acids with a chain length of 3 to 30 C atoms, and saturated and/or unsaturated, branched or unbranched alcohols with a chain length from 3 to 30 C atoms from the group of the esters from aromatic carboxylic acids, and saturated and/or unsaturated, branched and/or unbranched alcohols with a chain length from 3 to 30 C atoms. Such ester oils can then be advantageously selected from the group of isopropylmyristate, isopropylpalmitate, isopropylstearate, isopropyloleate, n-butylstearate, n-hexyllaureate, n-decyloleate, isooctylstearate, isononylstearate, isononylisononanoate, 2-ethylhexylpalmitate, 2-ethylhexyllaureate, 2-hexyldecylstearate, 2-octyldocecylpalmiate, oleyloleate, oleylerucate, erucylerucate, as well as synthetic, semisynthetic, and natural mixtures of such esters, for example, jojoba oil.

Furthermore, one may advantageously select the oil phase from the group of branched and unbranched hydrocarbons and hydrocarbon waxes, silicone oils, dialkylether, of the group of saturated or unsaturated, branched or unbranched alcohols, as well as the fatty acid triglycerides, namely the triglycerin ester of saturated and/or unsaturated, branched and/or unbranched alkanoic acids of a chain length from 8 to 24, in particular 12 to 18 C atoms. One may advantageously select the fatty acid triglycerides, for example, from the group of the synthetic, semisynthetic, and natural oils, for example, olive oil, sunflower oil, soy bean oil, peanut oil, rape seed oil, almond oil, palm oil, coconut oil, palm kernel oil, and more of the like.

Likewise any desired mixtures of such oil and wax components are to be advantageously used for the purposes of the present invention. If need be, it can also be advantageous to use waxes, for example, cetylpalmitate as the sole lipid component of the oil phase.

The oil phase is advantageously chosen from the group of 2-ethylhexylisostearate, octyldodecanol, isotridecyl isononanoate, isoeicosane, 2-ethylhexylcocoate, C₁₂-₁₅alkylbenzoate, caprylic/capric acid triglyceride, and dicaprylether.

Especially advantageous are mixtures of C₁₂₋₁₅-alkylbenzoate and 2-ethylhexylisostearate, mixtures of C₁₂₋₁₅-alkylbenozate and isotridecyl isononanoate, as well as mixtures of C₁₂₋₁₅-alkylbenzoate, 2-ethylhexylisostearate and isotridecylisononanoate.

Of the hydrocarbons, one can advantageously use for the purposes of the present invention, paraffinic acid, squalane and squalene.

Advantageously, the oil phase may further include a content of cyclic or linear silicone oils, or entirely consist of such oils. However, besides the silicone oil or oils, it is preferred to use an additional content of other oil phase components.

As a silicone oil that is to be used in accordance with the invention, one advantageously uses cyclomethicone (octamethyl cyclotetrasiloxane). However, other silicone oils can also be used advantageously for the purposes of the present invention, for example, hexamethyl cyclotrisiloxane, polydimethylsiloxane, and poly(methylphenylsiloxane).

Other particularly advantageous mixtures are those of cyclomethicone and isotridecyl isononanoate, of cyclomethicone and 2-ethylhexyl isostearate.

The aqueous phase of the preparations according to the invention advantageously comprises, if need be, alcohols, diols, or polyols having a low number of C atoms, as well as their ethers, preferably ethanol, isopropanol, polypropylene glycol, glycerin, ethyleneglycol, ethyleneglycol monoethyl- or -monobutyl ether, polypropylene glycolmonmethyl, -monoethyl-, or -monobutyl ether, diethyleneglycol monomethyl- or -monoethyl ether, and analogous products, furthermore alcohols having a low number of C atoms, for example, ethanol, isopropanol, 1,2-propanediol, glycerin, as well as in particular one or more thickeners, which may advantageously be selected from the group of silicon dioxide, aluminum silicates, polysaccharides, or their derivatives, for example, hyaluronic acid, xanthane gum, hydroxypropylmethyl cellulose, very advantageously from the group of the polyacrylates, preferably a polyacrylate from the group of so-called Carbopols, for example, Carbopols of the grades 980, 981, 1382, 2984, 5984, each alone or in combination.

In particular, mixtures of the aforesaid solvents are used. In the case of all alcoholic solvents, water may be a further ingredient.

Emulsions of the invention are advantageous, and they contain, for example, the foregoing fats, oils, waxes, and other lipids, as well as water and an emulsifier as is normally used for such a type of the formulation.

Gels according to the invention normally contain alcohols with a low number of C atoms, for example, ethanol, isopropanol, 1,2-propanediol, glycerin, and water, or an aforesaid oil in the presence of a thickener, which is in the case of oily-alcoholic gels preferably silicon dioxide or an aluminum silicate, in the case of aqueous-alcoholic or alcoholic gels preferably a polyacrylate.

Suitable as propellant for preparations according to the invention, that can be sprayed from aerosol containers, are the common, known, highly volatile, liquefied propellants, for example, hydrocarbons (propane, butane, isobutane), which can be used alone or mixed with one another. It is likewise advantageous to use compressed air.

Advantageously, preparations of the present invention may additionally contain substances, which absorb UV radiation in the UVB range, where the total amount of the filter substances is, for example, from 0.1 wt. % to 30wt. %, preferably 0.5 wt. % to 10 wt. %, in particular 1.0 to 6.0 wt. % based on the total weight of the preparations for making available cosmetic preparations, which protect hair and skin against the entire range of the ultraviolet radiation. They can also be used as sunscreen agents for the hair or skin.

When the preparations of the present invention contain UVB filter substances, same may be oil-soluble or water-soluble. In accordance with the invention, advantageous oil-soluble UVB filters include, for example:
derivatives of 3-benzylidene camphor, preferably 3-(4-methylbenzylidene) camphor, 3-benzylidene camphor;
derivatives of 4-aminobenzoic acid, preferably 2-(ethylhexyl)4-dimethylamino-benzoate, amyl 4(dimethylamino)-benzoate;
esters of cinnamic acid, preferably (2-ethylhexyl)4-methoxy cinnamate, isopentyl 4-methoxy cinnamate;
esters of salicylic acid, preferably (2-ethylhexyl) salicylicate, (4-isopropylbenzyl) salicylicate, homomenthyl salicylicate;
derivatives of benzophenone, preferably 2-hydroxy-4-methoxybenzophenone, 2-hydroxy-4-methoxy-4′-methylbenzophenone, 2,2′-dihydroxy-4-methoxybenzophenone;
esters of benzalmalonic acid, preferably di(2-ethylhexyl) 4-methoxybenzalmalonate; and -2,4,6-trianilino-(p-carbo-2′-ethyl-1′-hexyloxy)-1,3,5-triazine.

Advantageous water-soluble UVB filters are, for example:
salts of 2-phenylbenzimidazole-5-sulfonic acid, such as its sodium-, potassium-, or its triethanol ammonium salt, as well as the sulfonic acid itself;
sulfonic acid derivatives of benzophenones, preferably, 2-hydroxy-4-methoxybenzophenone-5-sulfonic acid and its salts; and
sulfonic acid derivatives of 3-benzylidene camphor, such as, for example, 4-(2-oxo-3-bornylidene-methyl)benzenesulfonic acid, 2-methyl-5-(2-oxo-3-bornylidenemethyl)sulfonic acid and its salts, as well as the 1,4-di(2-oxo-10-sulfo-3-bornylidene)benzene and salts thereof (the corresponding 10-sulfato compounds, for example the corresponding sodium-, potassium-, or triethanolammonium salt), also referred to as benzene-1,4-di(2-oxo-3-bornylidenemethyl-10-sulfonic acid).

Naturally, the list of the referenced UVB filters, which may be used in combination with the active ingredient combinations according to the invention, is not intended to be limiting.

It may also be of advantage to use in the preparations of the invention UVA filters, which are normally present in cosmetic preparations. These substances are preferably derivatives of dibenzoylmethane, in particular 1-(4′-tert-butylphenyl)-3-(4′-methoxyphenyl)propane-1,3-dione and 1-phenyl-3-(4′-isopropylphenyl)propane-1,3-dione. Likewise these combinations or preparations, which contain these combinations, are subject matter of the invention. The amounts used can be the same as the amounts for the UVB combination.

Cosmetic and dermatological preparations according to the invention may also contain inorganic pigments, which are normally used in cosmetics for protecting the skin against UV radiation. These pigments are oxides of the titanium, zinc, zirconium, silicon, manganese, cerium, and mixtures thereof, as well as modifications, wherein the oxides are the active agents. Very preferred are the pigments on the basis of titanium oxide.

Likewise, these combinations of UVA filter and pigment, or preparations containing this combination, are subject matter of the invention. The amounts used can be the same as the amounts for the foregoing combinations.

Advantageously, the cosmetic and dermatological preparations contain constituents and auxiliaries as are normally used for this type of preparations for the care and treatment of hair. Auxiliaries include preservatives, surface-active substances, antifoaming substances, thickeners, emulsifiers, fats, oils, waxes, organic solvents, bactericides, perfumes, dyes, or pigments, which are to dye the hair or the cosmetic or determatological preparation itself, electrolytes, substances that prevent hair from becoming oily.

For the purposes of the present invention, electrolytes are water-soluble alkali-, ammonium-, alkaline earth—(including magnesium), and zinc salts of inorganic anions and any mixtures of such salts. In this connection, it must be ensured that these salts be pharmaceutically and cosmetically safe.

It is preferred to select the anions of the invention from the group of the chlorides, sulfates, and hydrogensulfates, phosphates, hydrogenphosphates, and the linear and cyclic oligophosphates, as well as carbonates and hydrogencarbonates.

Cosmetic preparations, which are a skin cleansing agent, preferably contain at least one anionic, non-ionic, or amphoteric surface-active substance, or also mixtures of such substances, the combinations of active constituents as used in accordance with the invention in an aqueous medium, and auxiliaries as are normally used for this purpose. The surface-active substance or the mixtures of these substances may be present in the shampooing agent in a concentration between 1 wt. % and 50 wt. %.

These cosmetic or dermatological preparations may also be aerosols with the auxiliaries that are normally used for this purpose.

Aqueous cosmetic cleansing agents of the invention, or dry or anhydrous cleansing agent concentrates that are intended for aqueous cleansing, may contain anionic, non-ionic, and/or amphoteric surfactants, for example:
conventional soaps, for example, fatty acid salts of the sodium
alkyl sulfates, alkylether sulfates, alkane and alkyl benzene sulfates
sulfoacetates
sulfobetaines
sarcosinates
amidosulfobetaine
sulfosuccinates
half-esters of the sulfosuccinic acid
alkyl ether carboxylates
protein-fatty-acid condensates
alkyl betaines and amidobetaines
alkanolamides of the fatty acid
polyglycol ether derivatives

Cosmetic preparations, which are cosmetic cleansing preparations for the skin, may be present in liquid or solid form. They contain in addition to the active ingredient combinations of the invention, preferably at least one anionic, non-ionic, or amphoteric surface-active substance or mixtures thereof, if desired, one or more electrolytes and auxiliaries as are normally used for this purpose. The surface-active substance may be present in the cleansing preparations in a concentration between 1 wt. % and 94 wt. % based on the total weight of the preparations.

Besides the aforesaid surfactants, the compositions according to the invention contain water and, if need be, additives that are common in cosmetics, for example, perfume, thickener, dyes, deodorants, antimicrobial substances, replenishers, complexing and sequestering agents, pearlizers, plant extracts, vitamins, active substances, and the like.

For the preparations of the invention it is preferred to use forms of storage that permit little access of atmospheric oxygen. Thus, for example, it is advantageous to fill them in an inert gas atmosphere, in particular nitrogen. Especially advantageous for packaging are aluminum tubes.

The following examples are to illustrate the invention without limiting it. Unless otherwise indicated, all specified quantities, moieties, and percentages are based on the weight and the total quantity or the total weight of the preparations.

### EXAMPLE 1

| W/O Cream | Wt. % |
|---|---|
| Paraffin oil | 10.00 |
| Petrolatum | 4.00 |
| Wool wax alcohol | 1.00% |
| PEG-7 hydrated castor oil | 3.00 |
| Aluminum stearate | 0.40 |
| Ascorbic acid | 1.0 |
| Glycerin | 2.00 |
| Water, preservatives, and perfume | ad 100.00 |

### EXAMPLE 2

| W/O Lotion | Wt. % |
|---|---|
| Paraffin oil | 20.00 |
| Petrolatum | 4.00 |
| Glucose sesquiisostearate | 2.00 |
| Aluminum stearate | 0.40 |
| Ascorbic acid | 1.5 |
| Coenzyme Q10 | 0.1 |
| Vitamin E acetate | 2.00 |
| Vitamin C palmitate | 0.20 |
| Glycerin | 5.00 |
| Water, preservatives, and perfume | ad 100.00 |

### EXAMPLE 3

| Lip Care Stick | Wt. % |
|---|---|
| Hydrated castor oil | 4.00 |
| Ceresin | 8.00 |
| Beeswax | 4.00 |
| Carnauba wax | 2.00 |
| Petrolatum | 40.00 |
| Ascorbic acid | 2.0 |
| Coenzyme Q10 | 0.25 |
| β-Carotene | 0.10 |
| Paraffin oil, pigments, and dyes | ad 100.00 |

### EXAMPLE 4

| Liposome-containing gel | Wt. % |
|---|---|
| Lecithin | 6.00 |
| Shea butter | 3.00 |
| Ascorbic acid | 2.0 |
| Vitamin A palmitate | 0.20 |
| Biotin | 0.08 |
| Sodium citrate | 0.50 |
| Glycine | 0.20 |
| Urea | 0.20 |
| Sodium PCA | 0.50 |
| Hydrolyzed collagen | 2.00 |
| Xanthane gum | 1.40 |
| Sorbitol | 3.00 |
| Water, preservatives, and perfume | ad 100.00 |

### EXAMPLE 5

| W/O Cream | Wt. % |
|---|---|
| Paraffin oil | 10.00 |
| Petrolatum | 4.00 |
| Wool wax alcohol | 1.00% |
| PEG-7 hydrated castor oil | 3.00 |
| Aluminum stearate | 0.40 |
| Ascorbic acid | 0.5 |
| Coenzyme Q10 | 0.5 |
| Alpha-hydroxy palmitic acid | 0.50 |
| Glycerin | 2.00 |
| Water, preservatives, and perfume | ad 100.00 |

### EXAMPLE 6

| W/O Lotion | Wt. % |
|---|---|
| Paraffin oil | 20.00 |
| Petrolatum | 4.00 |
| Glucose sesquiisostearate | 2.00 |
| Aluminum stearate | 0.40 |
| Ascorbic acid | 1.0 |
| Alpha-hydroxy palmitic acid | 0.3 |
| Vitamin E acetate | 2.00 |
| Vitamin C palmitate | 0.20 |
| Glycerin | 5.00 |
| Water, preservatives, and perfume | ad 100.00 |

### EXAMPLE 7

| O/W Lotion | Wt. % |
|---|---|
| Paraffin oil | 8.00 |
| Isopropylpalmitate | 3.00 |
| Petrolatum | 4.00 |
| Cetearyl alcohol | 2.00 |
| PEG-40 castor oil | 0.50 |
| Sodium cetearylsulfate | 0.50 |
| Sodium carbomer | 0.40 |
| Phytic acid | 2.0 |
| Ascorbic acid | 0.5 |
| Glycerin | 3.00 |
| Alpha-tocopherol | 0.20 |
| Octylmethoxycinnamate | 5.00 |
| Butylmethoxydibenzoylmethane | 1.00 |
| Water, preservatives, and perfume | ad 100.00 |

### EXAMPLE 8

| O/W Cream | Wt. % |
|---|---|
| Paraffin oil | 7.00 |
| Avocado oil | 4.00 |
| Glyceryl monostearate | 2.00 |
| Sodium stearate | 1.00 |
| Ascorbic acid | 0.8 |
| Coenzyme Q10 | 0.25 |
| Deferoxamine | 0.2 |
| Sodium phytate | 1.00 |
| Alpha-hydroxy palmitic acid | 0.3 |
| Titanium dioxide | 1.00 |
| Sodium lactate | 3.00 |
| Glycerin | 3.00 |
| Water, preservatives, and perfume | ad 100.00 |

1. Use of ascorbic acid for producing topical preparations with angiogenic effects.

2. Use of claim 1, characterized in that the ascorbic acid is present in cosmetic or topical dermatological preparations in concentrations from 0.001 wt. % to 10 wt. %, preferably 0.05 wt. % to 5 wt. %, in particular 0.1 wt. % to 2.0 wt. % based on the total weight of the preparations.
